(19) United States
(12) Patent Application Publication
(10) Pub. No.: US 2012/0222682 A1
(43) Pub. Date: Sep. 6, 2012

(54) Tracheostomy Tube Securing System

(76) Inventors: Hanh Nguyen, Tempe, AZ (US); Sean Luong, Tempe, AZ (US)

(21) Appl. No.: 13/039,496

(22) Filed: Mar. 3, 2011

(51) Int. Cl. A61M 16/00

(52) U.S. Cl. 128/207.17

(57) ABSTRACT

A tracheostomy tube securer is disclosed herein comprising a band with a first set of apertures and a strap with a second set of apertures. Tracheostomy tube securer further comprises a band thread and a strap thread to lace through the sets of apertures. Tracheostomy tube securer further comprises a tab for quick release of the securing system in an emergency situation.

### BACKGROUND

The present invention relates generally to the field of tracheostomy tube (hereinafter “trach tube”) collars. A tracheostomy procedure consists of making an incision on the front of the neck and opening a direct airway through an incision in the trachea. The resulting hole, or “stoma”, can serve independently as an airway or as a site for a trach tube to be inserted; this tube allows a person to breathe without the use of his or her nose or mouth. The trach tube is inserted into the stoma and is commonly held in place by a device configured with straps or ties, often known as a trach collar. A tracheostomy is usually done for one of three reasons: (1) to bypass an obstructed upper airway (an object obstructing the upper airway will prevent oxygen from the mouth to reach the lungs); (2) to clean and remove secretions from the airway; and (3) to more easily, and usually more safely, deliver oxygen to the lungs. Some individuals, such as children and disabled adults, require a trach tube (and thus a trach tube collar) on a permanent or semi-permanent basis.

Adjusting to life with a trach tube and trach tube collar can be difficult for those who cannot survive without them. This is especially true in children and special needs adults. Trach tubes and trach tube collars must be changed throughout the day which can be uncomfortable for the wearer. Further, when the wearer is very young or mentally disabled, he or she will often try to remove the collar, which can cause the trach tube to fall out of the stoma. This can result in serious injury or death if a new, sterile trach tube is not immediately reinserted into the stoma. Additionally, a trach tube can become clogged with mucous or other secretions which impede the individual's breathing. In this situation, the trach tube must be immediately removed and replaced to restore normal breathing function. This requires releasing the trach tube from the trach tube collar or removing the trach tube collar entirely.

Several devices are known that allow a user to attach or otherwise secure a trach tube in a stoma. Among these are collars with straps that thread through side holes in the trach tube and tie or buckle in the back of the wearer's neck. Others utilize hook and loop closures on either side of the neck and/or in the back of the wearer's neck. Advantageously, all of these devices secure the trach tube in the stoma while at the same time permit the wearer or wearer's caregiver to easily attach and/or remove the collar or trach tube.

### BRIEF SUMMARY OF THE INVENTION

The known devices are not fully effective because they do not sufficiently prevent accidental or inappropriate removal by the wearer. This is because the collar typically is passively restrained by a simple hook and loop closure, giving the wearer the ability to remove the collar. This method can result in a young child or special needs adult inappropriately removing the collar thus risking serious injury or death. Passive hook and loop closures can also result in accidental removal if the collar becomes caught or snagged on a protruding object. While this method does allow the trach tube to be quickly removed in an emergency, such removal must be done at the expense of a secured fit. In the case of buckles, ties, or snaps, the fit is more secure, but takes much longer to remove. In an emergency situation, where seconds count, it is crucial that a new sterile trach tube be immediately inserted into the stoma to prevent serious injury or death. Fastening means such as buckles and ties can be cumbersome and difficult to undo quickly.

As individuals with trach tubes are typically incapable of speech, calling out for help is not an option in most cases should the trach tube fall out. Thus a need exists for a trach tube collar that is capable of being removed quickly in an emergency situation while at the same time preventing accidental or inappropriate removal. The present inventor has discovered that using a two part aperture fastening system with a quick release fastener in the back of the trach collar permits a trach collar to be securely fastened in a manner that prevents accidental or inappropriate removal while at the same time allowing for quick release in an emergency situation.

### BRIEF DESCRIPTION OF THE DRAWINGS

FIG. 1 consists of FIGS. 1A and 1B and illustrates a trach tube securing system embodying the principles of the present invention.

FIG. 2 consists of FIGS. 2A, 2B, 2C, and 2D and illustrates a front view of a disassembled trach tube securing system embodying the principles of the present invention.

FIG. 3 consists of FIGS. 3A and 3B and illustrates a side view of trach tube securing system and an internal view of an in-use condition.

FIG. 4 illustrates a method of using a trach tube securing system embodying the principles of the present invention.

### DETAILED DESCRIPTION

Referring now to the drawings in detail, this invention will be described by way of example and with reference to preferred embodiments thereof, however, it is to be understood that modifications and improvements may be made without departing from the scope or spirit of the invention. It should be noted that various changes and modifications to the presently preferred embodiments described herein will be apparent to those skilled in the art. Such changes and modifications may be made without diminishing its attendant advantages. It is, therefore, intended that such changes and modifications be included within the present invention. It should be understood that the current invention embodied herein is customizable and designed to work with any commercially available trach tube.

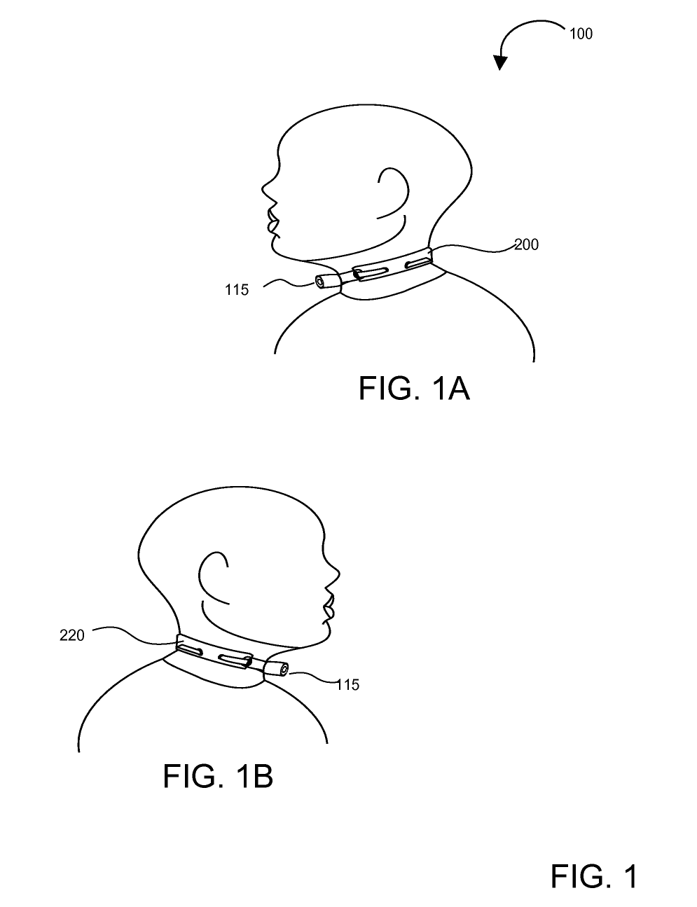

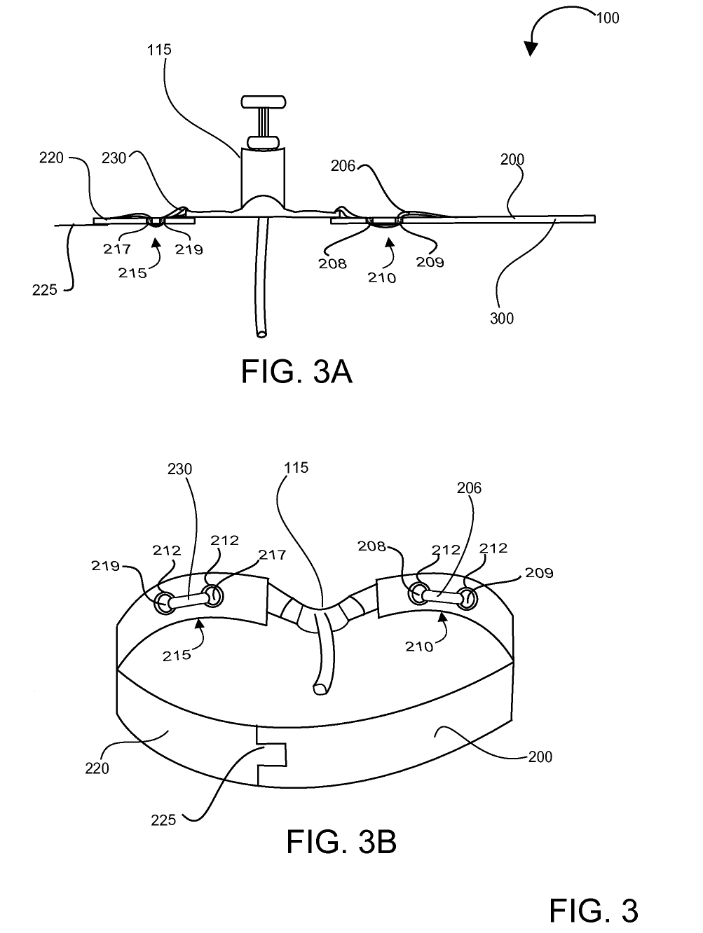

Referring now to FIG. 1, consisting of FIGS. 1A and 1B, and illustrating trach tube securing system 100 embodying the principles of the present invention. Trach tube securing system 100 may comprise band 200 and strap 220. Band 200 and strap 220 each may further comprise center layer 300 (shown in FIG. 3). Center layer 300 may comprise any pliant material sufficient to increase the durability and flexibility of band 200 and/or strap 220, such as foam rubber or a sponge-like material. Further, the top and or bottom of center layer 300 may be covered by any natural or synthetic material or fiber designed to be worn close to the skin such as cotton, nylon, rayon, or hemp. In this manner, center layer serves the purpose of providing flexibility of band 200 and/or strap 220, while the material surrounding center layer 300 may function as the outer and/or inner layer of band 200 and/or strap 220. In a preferred embodiment, center layer 300 is covered by a fiber that comprises a soft material sufficient to function as a nap in a hook and loop closure system, such as fleece or cotton. In this manner, band 200 and/or strap 220 may provide maximum comfort without sacrificing stability as trach tube securing system 100 is in use.

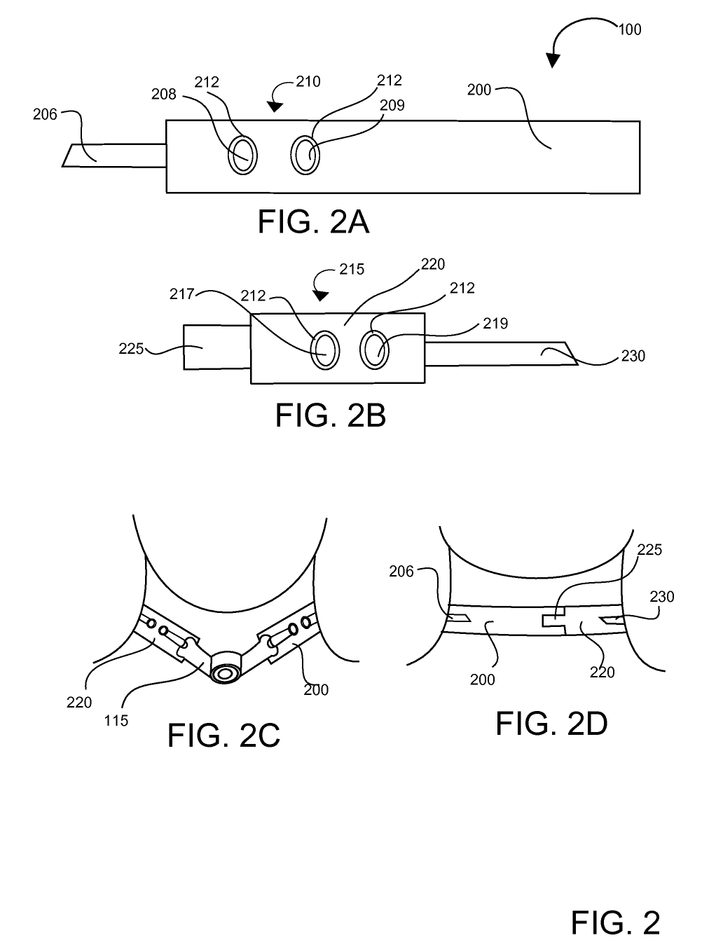

As shown in FIGS. 1A and 1B and discussed in further detail in FIGS. 2, 3, and 4, a portion of band 200 and strap 220 may be looped through either side of the front of trach tube 115 and attached to each other behind the wearer's neck. In this manner, trach tube 115 may be held securely in place via band 200 and/or strap 220. In alternative embodiments, band 200 and/or strap 220 may attach to each other on either side of the wearer's neck. As depicted in FIGS. 1A and 1B, band 200 is positioned on the left side of the wearer's neck while strap 220 is positioned on the right side. It should be understood that band 200 may be positioned on the right side of the wearer's neck while strap 220 may be positioned on the left. Further, trach tube securing system 100 is configured to encircle the wearer's neck to provide a secure, yet comfortable and easily removable collar for an individual's trach tube 115. In a preferred embodiment, trach tube securing system utilizes a standard hook and lope closure configuration for attachment to accomplish this purpose. However, it should be understood that other attaching means are capable of being used, including, but not limited to, adhesives, ties, buckles, snaps, pins and the like. It should further be understood that trach tube 115 is shown as an exemplary means only; trach tube securing system 100 may be customizable and is configured to accommodate and/or work in conjunction with any standard trach tube available and is not limited to the particular dimensions, shape, type, or brand of trach tube as depicted by numeral 115.

Referring now to FIG. 2, consisting of FIGS. 2A, 2B, 2C, and 2D, and illustrating a front view of band 200 and strap 220 embodying the principles of the present invention. FIG. 2 also depicts a front and back view of an in-use condition whereby the user is wearing trach tube securing system 100 to hold trach tube 115 in place. As mentioned above, trach tube securing system 100 comprises band 200. In a preferred embodiment, band 200 comprises a user-determined length sufficient to fit around a substantial portion of the neck of the trach tube wearer. It should be understood that the dimensions of band 200 are customizable and capable of being selected to accommodate the wearer's unique neck measurements. In a further preferred embodiment, band 200 comprises a soft material sufficient to provide the nap in a standard hook and loop closure configuration for attachment. However, it should be understood that other attaching means are capable of being used, including, but not limited to, adhesives, ties, buckles, snaps, pins and the like.

As depicted in FIG. 2A, band 200 may further comprise band thread 206. Band thread 206 may comprise an elongated strip that may be attached to the proximal or distal end of band 200 as shown. As depicted in FIG. 2A, band thread 230 is located on the proximal portion of band 200. It should be understood that band thread 206 may be located on the proximal or distal portion of band 200 according to the wearer's preference. In a preferred embodiment, the top-most portion of band thread 206 may comprise a series of hooks to function as the hook portion of a standard hook and loop closure configuration for attachment. However, it should be understood that other attaching means are capable of being used, including, but not limited to, adhesives, ties, buckles, snaps, pins, and the like.

Band 200 may further comprise first aperture set 210 configured to removably receive band thread. In a preferred embodiment, first aperture set 210 may comprise two (2) apertures as shown (discussed in further detail below). It should be understood that first aperture set 210 may comprise at least two apertures, but other combinations of apertures are contemplated without detracting from the spirit and scope of the present invention embodied herein. In a preferred embodiment, the proximal and distal apertures of first aperture set 210 may range from 1.0 to 2.0 centimeters apart. Further, in another preferred embodiment, the first proximal aperture of first aperture set may be 2.0 to 2.5 centimeters from the edge of band 200 as shown.

As mentioned above, band 200 further comprises first aperture set 210 which comprises at least two apertures. Within the present embodiment, when the user wishes to attach trach tube 115 using trach tube securing system 100, he/she or his/her caretaker may first insert trach tube 115 into the wearer's stoma (not shown). The wearer and/or his/her caretaker may then weave band thread 206 downward through first proximal aperture 208 and then up through first distal aperture 209 of first aperture set 210. Band thread 206 may then be pulled tight and pressed flat against band 200, leaving enough of band thread 206 to function as a pull tab, yet preventing inappropriate or accidental removal as illustrated in FIG. 2D. In this manner, band thread 206 works in conjunction with band 200 to function as a securing mechanism for trach tube 115.

As mentioned above, trach tube securing system 100 further comprises strap 220. In a preferred embodiment, strap 220 comprises a user-determined length sufficient to fit around the neck of the trach tube wearer while working in conjunction with band 200. It should be understood that the dimensions of strap 220 are customizable and capable of being selected to accommodate the wearer's unique neck measurements. In a preferred embodiment, strap 220 comprises a soft material sufficient to provide the nap in a standard hook and loop closure configuration for attachment. However, it should be understood that other attaching means are capable of being used, including, but not limited to, adhesives, ties, buckles, snaps, pins and the like.

As depicted in FIG. 2A, strap 220 may further comprise strap thread 230. Strap thread 230 may comprise an elongated strip that may be attached to the proximal or distal end of strap 220 as shown. As depicted in FIG. 2B, strap thread 230 is located on the proximal portion of strap 220. It should be understood that strap thread 230 may be located on the proximal or distal portion of strap 220 according to the wearer's preference. In a preferred embodiment, the top layer of strap thread 230 may comprise a hook portion (not shown) in order to stick to the soft material covering center layer 300 of strap 220 in the fashion of a hook and loop closure. However, it should be understood that other attaching means are capable of being used, including, but not limited to, adhesives, ties, buckles, snaps, pins and the like.

As alluded to above, strap 220 may further comprise second aperture set 215 configured to removably receive band thread. In a preferred embodiment, second aperture set 215 may comprise two (2) apertures as shown. It should be understood that second aperture set 215 may comprise at least two apertures (discussed in further detail below) but other combinations of apertures are contemplated without detracting from the spirit and scope of the present invention embodied herein. In a preferred embodiment, the proximal and distal apertures of second aperture set 215 may range from 1.0 to 2.0 centimeters apart. Further, in another preferred embodiment the first proximal aperture of first aperture set may be 2.0 to 2.5 centimeters from the edge of strap 220 as shown.

Within the present embodiment and in a similar manner as that described in paragraph [0016], the wearer may fasten strap 220 to trach tube 115. The wearer or wearer's caretaker may attach strap 220 to trach tube 115 by weaving strap thread 230 up and around the appropriate opening in trach tube 115 (on the opposite side of band 200 as illustrated in FIG. 2C). Strap thread 230 may then be threaded downward through second proximal aperture 217 and up through second distal aperture 219. Strap thread 230 may then be pulled tight and pressed flat against strap 220, leaving enough of strap thread 230 to function as a pull tab, yet preventing in appropriate removal by the wearer, as illustrated in FIG. 2D. In this manner, strap thread 230 works in conjunction with strap 220 to function as a securing mechanism for trach tube 115. It should be understood that the sequence of attaching band 200 and/or strap 220 are interchangeable, e.g., the user may opt to secure strap 220 to trach 115 before attaching band 200 and vice versa.

Strap 220 may further comprise tab 225. Tab 225 may comprise an elongated strip that may be attached opposite of strap thread 230 and to the distal or proximal end of strap 220. As depicted in FIG. 2B, tab 225 is attached to the distal portion of strap 220. It should be understood that tab 225 may be located on the proximal or distal portion of strap 220 according to the wearer's preference. In a preferred embodiment, the top layer of tab 225 may comprise a hook portion (not shown) in order to stick to the soft material covering center layer 300 of band 200 in the fashion of a hook and loop closure. However, it should be understood that other attaching means are capable of being used, including, but not limited to, adhesives, ties, buckles, snaps, pins and the like.

Once the wearer or wearer's caretaker has fastened band 200 and strap 220 in the manner described above, he or she may then encircle the wearer's neck with trach tube securing system 100. Tab 225 may be configured to be slightly smaller than band 200. When the wearer or wearer's caretaker wishes to fasten band 200 to strap 220, he or she may press tab 225 over the topmost portion of band 200 as depicted in FIG. 2D. In a preferred embodiment, tab 225 may be located at the back of the wearer's neck. In this manner, trach tube securing system is configured with an emergency quick-release mechanism. When the wearer or wearer's caretaker must quickly remove trach tube 115, he or she may unfasten trach tube securing system 100 by pulling on tab 225 to remove strap 220 from band 200.

It should be understood that in alternative embodiments, tab 225 may function to couple band 220 and strap 225 on any user preferred portion of the wearer's neck. In a further preferred embodiment, the bottom layer of tab 225 may comprise a hook portion (not shown) in order to stick to the soft material of band 200 in the fashion of a hook and loop closure. However, it should be understood that other attaching means are capable of being used, including, but not limited to, adhesives, ties, buckles, snaps, pins and the like. In this manner, tab 225 and strap 220 work in conjunction with band 200 to accomplish the task of securing trach tube 115 in place while at the same time providing the means for emergency/quick release of trach tube securing system from the wearer's neck.

As shown in FIGS. 2A and 2B, trach tube securing system 100 may further comprise at least one grommet 212 configured to reinforce first aperture set 210 and/or second aperture set 215. Grommet 212 may comprise a substantially circular reinforced eyelet. In this manner, grommet 212 functions to facilitate band thread 206 and/or strap thread 230 as they are woven through first aperture set 210 and/or second aperture set 215. Further, grommet 212 made be of any material sufficient to surround and protect first aperture set 210 and/or second aperture set 215 including, but not limited to, metal and metal alloys, plastics or other polymers, natural or synthetic rubber, or any other suitable material sufficient to provide reinforcement.

FIGS. 2C and 2D illustrate trach tube securing system 100 in an in-use condition whereby strap 220 is attached to band 200 via tab 225. In a preferred embodiment, all attaching means within trach tube securing system 100 comprise hook and loop closures. However, other fastening means are contemplated without departing from the scope and spirit of the present invention embodied herein, including, but not limited to snaps, buckles, buttons, hooks and eyes, or other suitable fastening means. Further, within the present figure shown, band 200 is depicted on the left side of the wearer's neck while strap 220 is depicted on the right side of the wearer's neck. It should be understood that band 200 and strap 220 may be attached to either side of trach tube 115. It should be further understood that the dimensions of band 200 and strap 220 are user customizable, e.g., the length of either is determined by the dimensions of the wearer's neck and trimmed from a precut length accordingly.

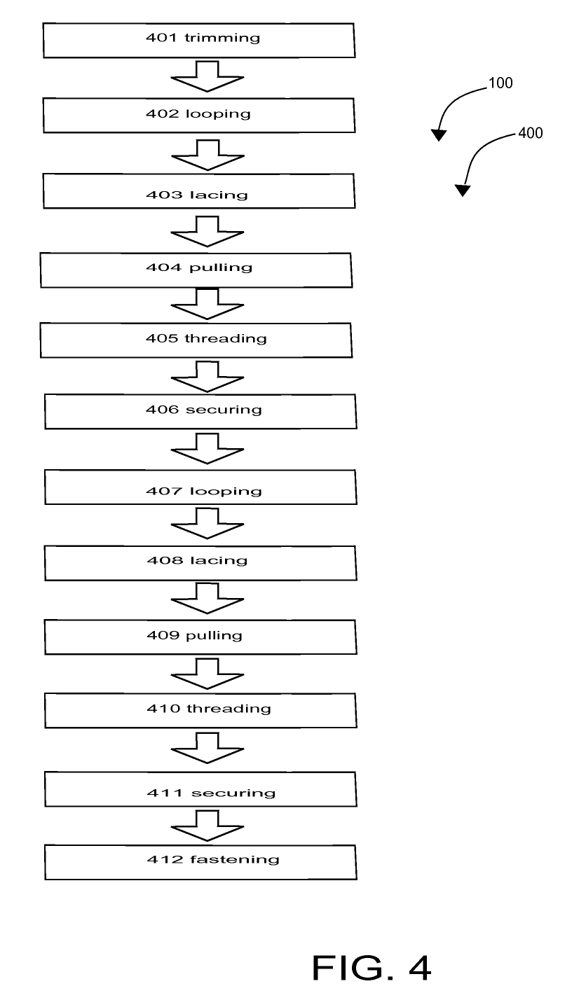

FIG. 3 consists of FIGS. 3A and 3B and illustrates a side view of trach tube securing system 100 and an internal view of an in-use condition. As depicted in FIG. 3A and discussed in FIGS. 2 and 4, band thread 206 may be looped through one side of trach tube 115 as shown. Band thread 206 may then be threaded through first proximal aperture 208 and first distal aperture 209 in first aperture set 210, respectively. In a preferred embodiment, the underside of band thread 206 comprises a hook closure configuration. In this manner, the hook portion of band thread 206 fastens to the proximal end of the pliant material of the first proximal aperture 208 within band 200. Similarly, strap thread 230 may be looped through the opposite side of trach tube 115. As depicted in FIG. 3A and discussed in FIGS. 2 and 4, strap thread 230 may be looped through one side of trach tube 115 as shown. Strap thread 230 may then be threaded through second proximal aperture 217 and second distal aperture 219 in second aperture set 215, respectively. In a preferred embodiment, the underside of strap thread 225 comprises a hook closure configuration. In this manner, the hook portion of strap thread 230 fastens to the proximal end of the pliant material of the second proximal aperture 219 within strap 220. As depicted in FIG. 3A, band thread 206 and strap thread 230 operate in conjunction on either side of trach tube 115 to hold trach tube 115 in place securely while preventing accidental or improper unfastening.

Once strap thread 230 and band thread 206 are sufficiently holding trach tube 115, trach tube securing system 100 may be fastened around the neck of the wearer by affixing the hook portion of tab 225 to the distal portion of band 200 as depicted in FIG. 3B. As a result, if there is an emergency situation requiring quick removal of trach tube 115, the wearer or wearer's caretaker may undo the hook closures of tab 225 from loop closures of band 200 for quick release and removal of trach tube 115. In this manner, band 200 and strap 220 remain fastened to trach tube 115 thus eliminating the requirement that secure fastening of trach tube 115 be sacrificed in order to accommodate emergency removal.

FIG. 4 is a flowchart illustrating a method of use 400 for trach tube securing system 100 embodying the principles of the present invention. Method of use 400 for operating trach tube securing system 100 preferably comprises the steps of: step one 401 trim the length of band 200 length to accommodate the unique dimensions of the wearer's neck; step two 402 loop band thread 206 through the appropriate portion of trach tube 115; step three 403 lace band thread 206 through first proximal aperture 208 of first aperture set 210; step four 404 pull band thread 206 tightly through first proximal aperture 208 of first aperture set 210; step five 405 thread band thread 206 up through the first distal aperture 209 of first aperture set 210; step six 406 secure the remaining portion of band thread 206 against band 200; step seven 407 loop strap thread 230 through the appropriate portion of trach tube 115 (on the opposite side of the currently threaded band thread 206); step eight 408 lace strap thread 220 through the first hole of second aperture set 215; step nine 409 pull strap thread 230 tightly through the second proximal aperture 217 of second aperture set 215; step ten 410 thread strap thread 230 up through the second distal aperture 219 of second aperture set 215; step eleven 411 secure the remaining portion of strap thread 230 against strap 220; and step twelve 412 fasten trach tube securing system 100 around wearer's neck by attaching tab 225 to band 200.

Method of use 400 further comprises the optional steps (not shown) of removing trach tube securing system comprising: step 413 removing band thread 206 from band 200; step 414 unthreading band thread 206 from first distal aperture 209; step 415 unthreading band thread 206 from first proximal aperture 208; step 416 unthreading band thread 206 from trach tube 115; step 417 removing strap thread 230 from strap 220; step 418 unthreading strap thread 230 from second distal aperture 219; step 419 unthreading strap thread 220 from second proximal aperture 217; and step 420 unthreading strap thread 230 from trach tube 115. Finally, method of use 400 further comprises the optional step (not shown) of removing trach tube securing system comprising: step 421 separating band 200 from strap 200 by unfastening tab 225.

It should be noted that the steps described in the method of use can be carried out in many different orders according to user preference. Upon reading this specification, it should be appreciated that, under appropriate circumstances, considering such issues as design preference, user preferences, marketing preferences, cost, structural requirements, available materials, technological advances, etc., other methods of use arrangements such as, for example, different orders within above-mentioned list, elimination or addition of certain steps, including or excluding certain maintenance steps, etc., may be sufficient.

From the foregoing description, it should be appreciated that a preferred embodiment and a method for securing a trach tube is provided and present significant benefits that would be apparent to one skilled in the art. Furthermore, it should be appreciated that a vast number of variations in the embodiments exist. The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientists, engineers, and practitioners in the art who are not familiar with patent or legal terms or phraseology, to determine quickly from a cursory inspection the nature and essence of the technical disclosure of the application. It being understood that various changes may be made in the function and arrangement of elements described in the exemplary preferred embodiment without departing from the spirit and scope of the invention as set forth in the appended claims.

The foregoing descriptions of specific embodiments of the present invention have been presented for purposes of illustration and description. They are not intended to be exhaustive or to limit the present invention to the precise forms disclosed, and obviously many modifications and variations are possible in light of the above teaching. The exemplary embodiment was chosen and described in order to best explain the principles of the present invention and its practical application, to thereby enable others skilled in the art to best utilize the present invention and various embodiments with various modifications as are suited to the particular use contemplated.

What is claimed is new and desired to be protected by Letters Patent is set forth in the appended claims:

1. A tracheostomy tube securer comprising:
at least one band having a band thread and at least one first set of apertures;
at least one strap having a strap thread and at least one second set of apertures;
wherein said band thread connects to said band via said set of apertures; and
wherein said strap thread connects to said strap via said second set of apertures.

2. The band of claim 1 wherein said band further comprises at least one center layer comprising soft material.

3. The band of claim 2 wherein said center layer further comprises a pliant material.

4. The band of claim 1 wherein said band thread loops through a tracheostomy tube.

5. The band of claim 1 wherein said band thread loops through said at least one first set of apertures.

6. The strap of claim 1 wherein said band further comprises at least one center layer comprising soft material.

7. The strap of claim 6 wherein said center layer further comprises a pliant material.

8. The strap of claim 1 wherein said strap thread loops through a tracheostomy tube.

9. The strap of claim 1 wherein said strap thread loops through said at least one second set of apertures.

10. The strap of claim 1 wherein said strap thread fastens to said strap.

11. The strap of claim 1 further comprising at least one tab.

12. The strap of claim 12 wherein said at least one tab attaches to said at least one band to said at least one strap.

13. A tracheostomy tube securing system comprising:
a tracheostomy tube securing apparatus,
having at least one band comprising at least one center layer, said at least one band having a band thread, at least one strap having a strap thread and a tab, a first set of apertures and looping through at least one tracheostomy tube to attach said tracheostomy tube to a wearer.
said band thread loops through said tracheostomy tube;
said band thread attaches to said band;
said strap thread loops through said tracheostomy tube;
said strap thread attaches to said strap;
said tab attaches said strap to said band;
a securing system in connection with said tracheostomy tube to secure said tracheostomy tube to said wearer;
wherein said band provides a secure hold of said tracheostomy tube;
wherein said strap provides a secure hold of said tracheostomy tube; and
wherein said tab attaches band to strap.

14. The tracheostomy tube securing system of claim 14 further comprises a kit including said tracheostomy tube securing system; and a set of user instructions.

15. A method of operating a tracheostomy tube securing system comprising the steps of:
trimming said band;
looping said band thread said trach tube;
lacing said band thread through said first set of apertures;
pulling said band thread tightly through said first set of apertures;
securing said band thread to said band;
looping said strap thread through said trach tube on the opposite side said band thread;
lacing said strap thread said second set of apertures;
pulling said strap thread tightly through said second set of apertures;
securing said strap thread to said strap; and
attaching said strap to said band via said tab.

16. The method of operating a tracheostomy tube securing system of claim 16 further comprising the step of unthreading said band from said first set of apertures.

17. The method of operating a tracheostomy tube securing system of claim 16 further comprising the step of unthreading said strap from said second set of apertures.

18. The method of operating a tracheostomy tube securing system of claim 16 further comprising the step of unfastening said tab from said band.
